(19) United States
(12) Patent Application Publication
(10) Pub. No.: US 2023/0191049 A1
(43) Pub. Date: Jun. 22, 2023

(54) SYSTEM FOR CONTROLLING OF SELF-INFLATING RESUSCITATOR

(71) Applicants: UNITRA SP. Z O.O., Warszawa (PL); FUNDACJA BADAN I ROZWOJU NAUKI, Warszawa (PL)

(72) Inventors: Adrian KRUPOWICZ, Brwinow (PL); Michal WSZOLA, Warszawa (PL); Dominik DROBINSKI, Warszawa (PL); Przemek KITOWSKI, Gdansk (PL)

(73) Assignees: UNITRA SP. Z O.O., Warszawa (PL); FUNDACJA BADAN I ROZWOJU NAUKI, Warszawa (PL)

(21) Appl. No.: 17/995,979

(22) PCT Filed: Nov. 18, 2020
(86) PCT No.: PCT/PL2020/050083

§ 371 Date: Oct. 11, 2022

(30) Foreign Application Priority Data
Apr. 10, 2020 (PL) P.433528

(51) Int. Cl. A61M 16/00; A61M 16/20

(52) U.S. Cl. CPC A61M 16/0084; A61M 16/024; A61M 16/0051; A61M 16/201; A61M 2205/584; A61M 2205/581; A61M 2205/3331

(57) ABSTRACT

The self-inflating bag control system is characterized in that it comprises a bag controlling valve which is connected to at least two gas pumps and at least one gas parameter sensor via the gas distribution tubes. It comprises at least one gas pressing device connected to the valve via a gas parameter sensor via gas flow tubes. A control unit with a control panel is connected to the system through a gas sensor, which includes a sub-unit for setting the parameters of the gas flowing into the self-expanding bag, a measuring and analyzing the parameters of the gas flowing in the system by measuring the pressure and volume of the gas from the as parameters sensor, and a warn-alarm sub-unit, which generates a warning and/or alarm signal on the basis of the gas parameters read from the measurement and analysis subunit.

The invention relates to a self-inflating bag control system, commonly known as Ambu.

A self-inflating bag (resuscitator) is a device used in resuscitation of a non-breathing or ineffective breathing patient, allowing ventilation with high concentrations of oxygen.

It is connected with a mask worn over the patient's mouth and nose or with an endotracheal tube.

Without an additional supply of oxygen, the self-inflating bag enables ventilation with atmospheric air, i.e. oxygen at a concentration of 21%. After connecting oxygen with a flow of 5-6 l/min to a bag, the oxygen concentration increases to 45%, after increasing the flow to 10 l/min and using an additional reservoir, it is possible to obtain a concentration of approx. 85%.

It is necessary to manually control the resuscitators to resuscitate the patient in emergency cases when the patient's breathing is insufficient (respiratory failure) or has stopped completely (respiratory arrest). This can be done by professional paramedics.

Solutions are being sought to ensure effective control of the resuscitator—a self-inflating bag—for an effective, more automatic and accurate oxygen rescue.

The invention aims to replace the manual operation of compressing the bag, which in effect provides an autonomous device that supports the patient's breathing and works as a respirator in its simplest form.

The invention is based on the use of using with self-inflating bag and a system for automatic and controlled control of the gas flow to the resuscitator and the control of gas frequency and volume. A self-inflating bag control system is characterized by the fact that it comprises a valve controlling the flow of gas into a self-inflating bag that is connected to at least two gas pump and at least one gas parameters sensor through the gas distribution tubes, at least one gas pumping device, preferably in the form of a bellow, connected to the valve via gas parameter sensor via gas flow tubes. Additionally, the system is connected to the self-inflating bag by the gas flow tubes through the gas pumping device. A control unit with a control panel is connected to the system through a gas sensor, which comprises: a sub-unit for determining the parameters of the gas flowing into the self-inflating bag; sub-unit for measuring and analyzing the parameters of the gas flowing in the system by measuring the pressure and volume of the gas with a gas parameter sensor; a warn-alarm subunit which generates a warning and/or an alarming signal based on the readings of gas parameters from the measurement and analysis subunit.

An alarm signal is generated in the case of detection of system disorder, and a warning signal in the case of detection of a system malfunction.

On the control panel there are blue or green light generator, red light generator and yellow light generator, which generate respectively:

red light when a respiratory system disorder is detected,
yellow light when a system malfunction is detected,
blue or green light when a controlled operation of the system is detected.

Preferably the system includes: one gas valve, three gas pumps, two gas pumping devices, two gas parameters or gas pump sensors, three gas parameter sensors

Preferably, a pressure sensor for measuring the pressure parameters in the airway of the test subject connected to the control unit. Preferably, system comprises two gas valves, two gas pumps, two gas pumping devices, two gas parameter sensors, one pressure sensor

Preferably if the safe range of airway pressure is exceeded, an alarm signal in the form of light is generated in the warn-alarm subunit.

Preferably, in addition to an alarm or warning light signal, an audible signal is given—generated. Preferably, the acoustic signal is given as two beeps every 15-30 seconds or three beeps every 6-8 seconds for a warning signal and/or as ten beeps every 1-3 seconds for a disturbance signal.

The invention is shown more closely in the exemplary embodiments and in the drawing, in which:

Described in Example 1:

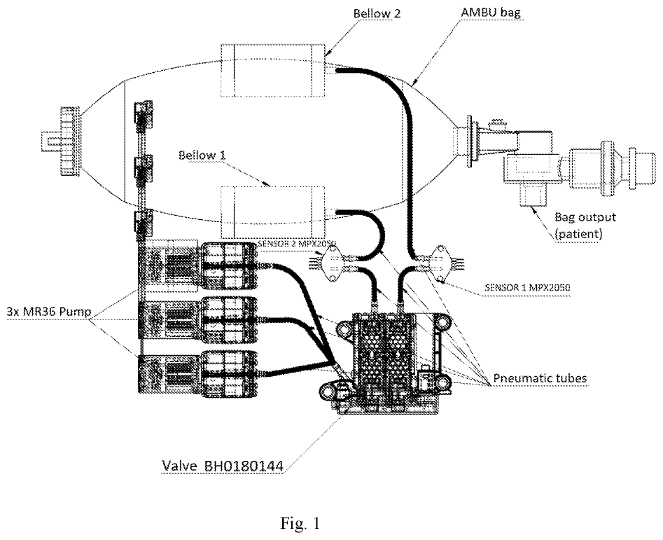

FIG. 1 a system according to the invention connected to a self-inflating bag

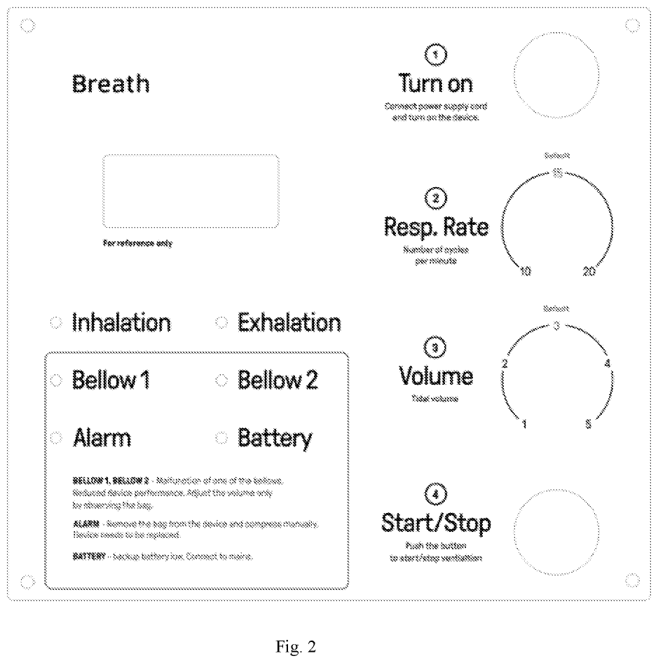

FIG. 2 control panel

Described in Example 2:

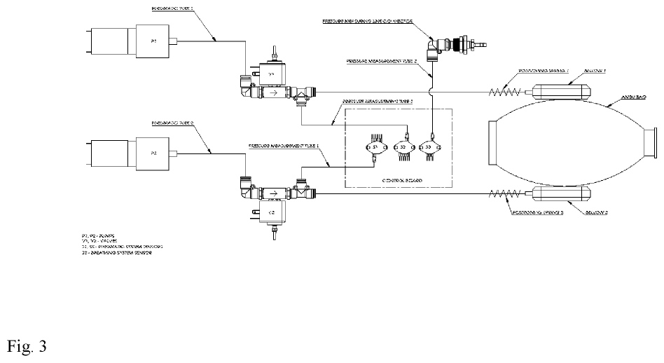

FIG. 3 extended system according to the invention

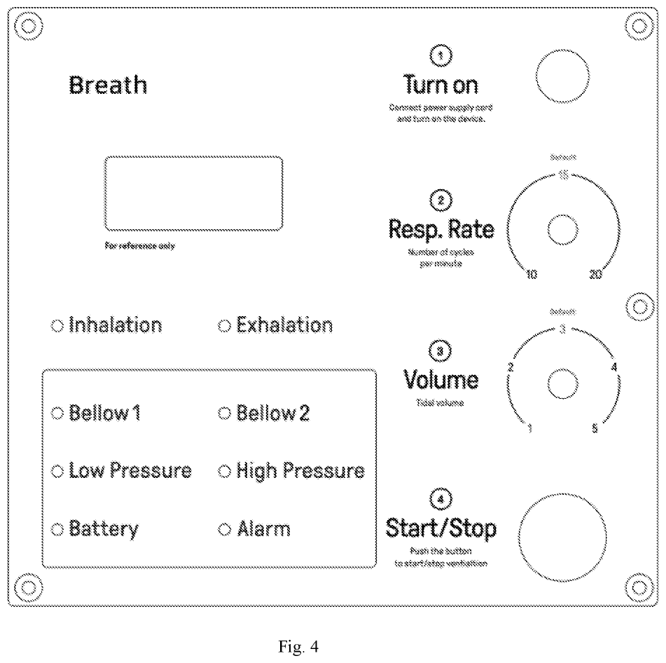

FIG. 4 extended system control panel And additional explanations on the application of the invention shown:

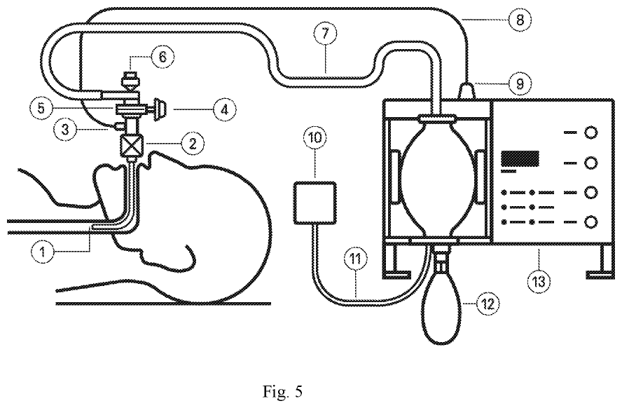

FIG. 5 application data of the system according to the invention in a variant of the overall system.

### EXAMPLE 1

As shown in FIG. 1, the system includes a valve that controls the flow of gas, e.g., air, directly to the patient via a self-inflating bag which is connected to the gas pump and gas sensors via tubing. There must be at least two pumps and at least one sensor.

A device in the form of a bellows for air pressure is connected to the valve through gas parameter sensors—a gas volume and pressure sensor. There are two sensors in the example, but there must be at least one. The sensors are connected to the valve and bellows via tubes. The tubes connect the system components and are used to distribute air to the self-inflating bag. The tubes connect the system with the self-inflating bag through a bellows connected with the bag by tubes.

A control unit is connected to the system through a sensor, thanks to that the system enables reading parameters from each part of the system (particularly reading from the gas pressure sensor), setting gas parameters and generating warning and alarm messages through the control unit.

This system enables the measurement and selection of the frequency and volume of gas flow to the self-inflating bag and, at the same time, alarming of incorrect operation and disturbed operation requiring operator control. Controlled parameters are pressure and gas volume in the system tubing.

The control unit has a control panel—with a management display to control the functionality of the system, which in the exemplary embodiment is soldered on the gold pins and shown in FIG. 2.

The control unit has 3 independent main control blocks:

analog control sub-unit, i.e. a sub-unit for setting the parameters of the gas flowing into the self-expanding bag;
a reference measurement sub-unit (measuring and analyzing sub-unit), analyzing the parameters of the gas flowing in the system by measuring the pressure and volume of the gas from the gas parameters sensor;
analog alarm sub-unit, i.e. warn-alarm sub-unit.

Analog control subunit (no programmable systems)—controls the operation of pumps and valves according to the rate and volume settings selected by the operator.

Digital sub-unit performing reference measurements (measurement and analysis)—the system calculates the current operation of the system and the parameters of the gas supplied to the bag. This subunit is connected to pressure sensors.

The analog warn-alarm sub-unit directly receives signals from the pressure sensors through the measuring and analyzing sub-unit and in the event of any failure it generates an alarm signal through a warning signal about reduced device performance, or a critical signal in the event of a system stoppage.

Using the control unit, readings from the gas sensor are analyzed—this unit allows you to control the frequency of gas supply, the volume of gas supply and the reception of warning or alarm signals—in the event of a failure or malfunction. Work of gas flow to the bag—the listed features are selected based on the read information of pressure and volume parameters—sensor and device measurement. A critical reading result turns on an alarm signal, and a warning reading result turns on a warning signal, which is signaled on the control panel with the appropriate colored LEDs. A red light generator, a yellow, blue or green light generator are used. A red light is generated when a system malfunction is detected, a yellow light when a system malfunction is detected, and a green/blue light, when a controlled system operation is detected.

A warning signal informs about a single fault, but a retained performance critical to the health of the patient. The second category is Critical. Serious dysfunction causing an alarm signal requiring immediate response from medical personnel and/or equipment shutdown.

The warning messages on the control panel with display are in accordance with the standard IEC 60601-1-8: 2006/AMD1: 2012 and are divided into categories. A cautionary sign informing about a single defect, but maintained efficiency critical to the patient's health.

The second category is Critical. The occurrence of a serious dysfunction causing the device to stop working and requiring immediate reaction of medical personnel.

Two-color colors are used to visualize the dysfunction: yellow and red.

Additionally, an acoustic signal is given: 2 beeps every 15-30 seconds (preferably 30) or 3 beeps every 6-8 seconds, preferably 7.5 seconds for a warning signal—incorrect operation of the system—signal from the yellow LED/indicator or 10 beeps every 1-3 seconds, preferably 2.5 seconds, with a signal about a work disturbance—a red LED. It was found that such a frequency is the most effective.

References to FIG. 2:

Color light indicators:
Blue/green:
   Turn on—signaling readiness to work on power;
   Start/Stop—signaling the start of ventilation by illuminating the button;
   Inhale/Exhale—signaling the operating status of the device by alternately lighting up;
Yellow/red—signaling alarm states in the device

### Battery:

Low priority—switches to power from a backup battery.
Medium priority—backup battery voltage below 11.5 V.
The time between the complete loss of power and the complete shutdown of the device is 40 minutes.

### Bellow 1, Bellow 2:

Medium Priority—Set tidal volume or rate does not reach the set value by more than 20%.

### Alarm:

High priority—device failure, pneumatic system not working. Immediately remove the bag from the device and squeeze it by hand

The system connected to the bag is shown in FIG. 1

In the exemplary embodiment, 3 pumps of the MR36 type are used, placed at the beginning of the system, which do not operate at full efficiency in normal operation. At least two pumps are required. Each pump has a diagnostic system. In case of failure of one pump, the device is still fully operational, but gives an indirect alarm to the control unit.

Two airborne air targets were used to ensure optimal operation. Pressure sensors (Sensor 1 and 2) type MPX2050, placed in the system downstream of the valve type BH0180144, constantly test the efficiency of the system. In case of failure of one of the cells, the device is able to support the modes required for minimum efficiency. The valve separately controls the two cells by means of airflow opening coils. Each chamber has a separate drain and air injection valve. Same as in the case of air targets. The malfunction of the valves is detected by the air flow sensors.

### EXAMPLE 2

As shown in FIG. 3, the system includes all the components as described in Example 1, except that it includes two pumps and three sensors.

The device for forcing air is in the form of bellows.

The control unit has a control panel which, in an embodiment, is soldered on the gold pins and shown in FIG. 4.

In the embodiment, two pumps (P1, P2) of the type RFP37G05R are used, placed at the beginning of the system. Each pump has a diagnostic system. If one pump fails, only one bellows will operate, the injected volume will be limited and gives an indirect alarm to the control unit.

In an embodiment of the invention, the system additionally includes at least one pressure sensor connected to the control unit, the sensor being connected to the patient's breathing circuit and monitoring the pressure in the airway. If the safe range of airway pressure is exceeded, the warning and alarm subunit will generate an alarm signal in the form of light and sound. Shown in FIG. 5.

Pressure sensors (S1, S2) type MPX5050GP placed in the system downstream of the valve type P8034, constantly test the efficiency of the system. In case of failure of one of the cells, the device is able to support the modes required for minimum efficiency. There are 2 independent valves (V1. V2) to control the air flow. Each chamber has a separate drain and air injection valve. The failure of the valves is detected by the pressure sensor and the alarm system.

Reference to FIG. 4:

Color light indicators:
Blue/green—same as in example 1
Yellow/red—same as in example 1 and in addition:
   Low pressure:
      Medium priority—airway pressure has dropped below 4 cmH2O
   High pressure:
      High priority—airway pressure has risen above 40 cmH2O.

Reference to FIG. 5:
1) Airway maintenance device:
   a) Laryngeal mask airway (LMA)
   b) Endotracheal tube (ETT)
2) Bacteria-viral filter (HMEF)
3) Pressure sensor
4) PEEP valve
5) Expiratory valve
6) Hypertensive valve
7) Breathing tube
8) Pressure measurement line
9) Pressure line connection
10) Oxygen Source:
   a) oxygen tank
   b) oxygen administered after pressure reduction
11) Oxygen tube
12) Enrichment bag
13) System

1. System for controlling of resuscitator in the form of a self-inflating bag, characterized in that, it comprises a gas flow control valve which is connected to at least two gas pumps and at least one gas parameter sensor via gas distribution pipes, at least one gas supply device which distributed gas to a self-inflating bag, preferably in the form of a bellow, connected to the valve through a gas parameter sensor through gas flow pipes, and in addition to gas flow pipes, the system during controlling is connected to a self-inflating bag through a gas pumping device, and a control unit for controlling gas parameters with a control panel is connected to the system through a gas sensor, which includes a subunit for determining the parameters of the gas flowing into the self-inflating bag, the subunit for measuring and analyzing the parameters of the gas flowing in the system by measuring the pressure and volume of the gas from the gas parameters sensor and the warning and alarming subunit, which, based on the readings of the gas parameters from the subunit the analyzer generates a warning and/or alarming signal, the alarming signal is generated in the event of a system malfunction and causes the system stoppage, and a warning signal in the event of detecting a system malfunction, and additionally in the control panel a red light generator and a yellow light generator is generating red light when a system malfunction is detected, yellow light when a system malfunction is detected, and a different cool color generator, preferably green or blue, is generating cool color when a controlled system operation is detected.

2. System according to claim 1, wherein it comprises one gas valve, three gas pumps, two gas supply devices.

3. The system according to claim 1, wherein it comprises two gas parameter sensors.

4. System according to claim 1, wherein it comprises two gas pumps and three gas parameter sensors.

5. System according to claim 1, wherein it comprises a pressure sensor for measuring the pressure parameters in the airway of the tested person connected to the control unit.

6. System according to claim 1, wherein it comprises two gas valves, two gas pumps, two gas pressure devices, two gas parameter sensors and one pressure sensor.

7. System according to claim 1, wherein when the safe range of airway pressure is exceeded, an alarm signal in the form of light is generated in the warning and alarming subunit.

8. System according to claim 1, wherein in addition to an alarm or warning light signal, an audio signal is transmitted.

9. System according to claim 8, wherein the audible signal is given as two beeps every 15-30 seconds or three beeps every 6-8 seconds for a warning signal and/or as ten beeps every 1-3 seconds for a disturbance signal.
